(19) United States
(12) Patent Application Publication
(10) Pub. No.: US 2008/0051476 A1
(43) Pub. Date: Feb. 28, 2008

(54) Alcohol production means

(76) Inventors: Matthew F. Russell, Spokane, WA (US); James John Moore, Gilbert, AZ (US)

Correspondence Address: Reidlaw LLC, 1926 South Valleyview Lane, Spokane, WA, 99212, US

(21) Appl. No.: 11/508,416

(22) Filed: Aug. 23, 2006

(51) Int. Cl. C07C 27/06

(52) U.S. Cl. 518/718

(57) ABSTRACT

In accordance with one embodiment of the present disclosure, a method of producing alcohol includes dispersing a catalyst in a supercritical fluid and causing carbon monoxide and hydrogen to contact the dispersed catalyst, whereby a reaction occurs to produce the alcohol.

### BACKGROUND

Synthesis gas (a.k.a. “syngas”) is a term known to those skilled in the art. Synthesis gas can be produced from many organic/carbonaceous sources such as, but not limited to, coal, petro-coke, municipal solid waste (“MSW”), refuse derived fuel (“RDF”), biogas from a digester, sewage sludge, corn and/or grain stover, switch grass, timber, grass clippings, construction demolition materials, cotton gin waste, biomass, landfill gas, natural gas, various types of animal and agricultural waste such as manure, and the like.

Various means for producing one or more types of alcohol from synthesis gas are also known to those of ordinary skill in the art. For example, one prior art method for producing alcohol from synthesis gas is disclosed by U.S. Pat. No. 6,753,353 to Jackson et al. According to the method taught by Jackson et al., a catalyst is suspended in liquid solvents to form a liquid slurry. The synthesis gas is then exposed to this liquid slurry to cause a reaction in which one or more types of alcohol can be formed.

### DESCRIPTION OF THE DRAWINGS

FIG. 1 is a phase diagram depicting various phases of an exemplary substance.

FIG. 2 is a schematic diagram depicting an apparatus in accordance with one embodiment of the present disclosure.

FIG. 3 is a schematic diagram depicting an apparatus in accordance with another embodiment of the present disclosure.

### DETAILED DESCRIPTION

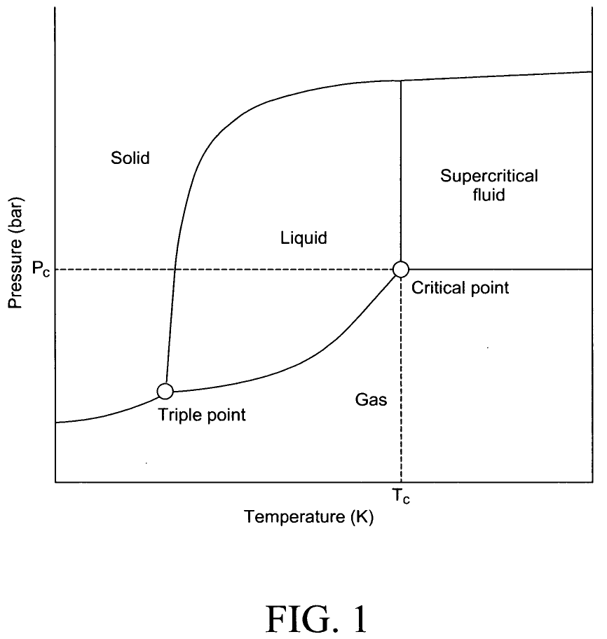

With reference to the drawings, FIG. 1 is a phase diagram for an exemplary substance in accordance with one embodiment of the present disclosure. It is to be understood that the phase diagram depicted by FIG. 1 is provided only for illustrative purposes and is not intended to be representative of any particular substance. Specifically, the phase diagram depicted by FIG. 1 is provided substantially only for the purpose of illustrating the concept of a “supercritical phase” for any given substance.

It is to be further understood that the given substance can be any substance that can exist in a supercritical phase, or as a supercritical fluid. By way of example only, it is understood that the given substance can be an element, a compound, or a mixture of elements and/or of compounds. Moreover, it is understood that the given substance can be that which exists under normal ambient atmospheric conditions as a gas, or that which exists under normal ambient atmospheric conditions as a liquid, or that which exists under normal ambient atmospheric conditions as a solid.

With continued reference to FIG. 1, temperature of the given substance is indicated by the horizontal axis, while pressure of the given substance is indicated by the vertical axis. As is seen from a study of FIG. 1, the given substance has an associated critical temperature that is indicated by T_{C}. The given substance also has an associated critical pressure that is indicated by P_{C}.

Continued study of FIG. 1 reveals that the given substance can exist as a “supercritical fluid” when both the temperature of the given substance is above the associated critical temperature T_{C}, and the pressure of the given substance is above the associated critical pressure P_{C}. Thus, a commonly accepted definition of a supercritical fluid is any substance that is above its thermodynamic critical point. By way of example, any of a number of known substances such as, but not limited to, hydrogen, carbon monoxide, carbon dioxide, water, ethanol, methanol, and the like, can exist as a supercritical fluid.

A given substance in a supercritical phase is generally much less dense than the given fluid in a liquid phase, and much more dense than the given substance in a gas phase. As is more specifically described below, we have discovered that this special characteristic of a supercritical fluid can be used advantageously in certain catalytic conversion processes to result in higher reaction rates and/or more efficient reactions for given reactants compared with prior art catalytic conversion processes.

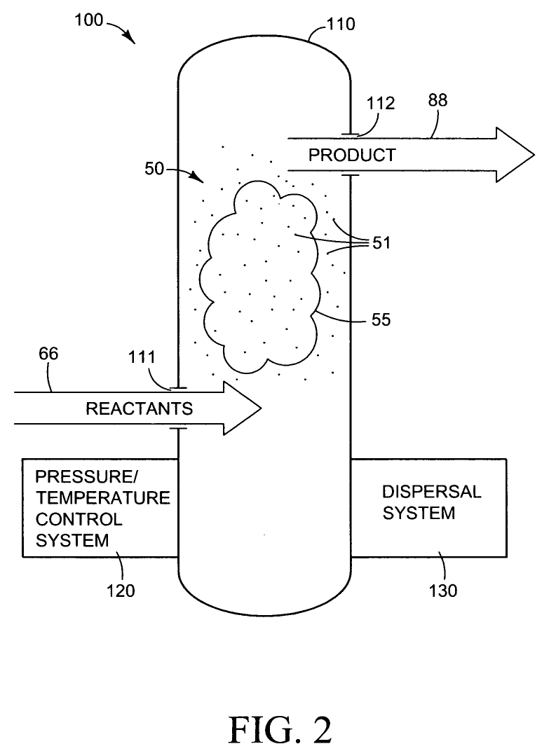

Turning now to FIG. 2, a schematic diagram is shown in which an apparatus 100 is depicted in accordance with at least one embodiment of the present disclosure. The apparatus 100 includes a reactor vessel 110. The reactor vessel 110 can be configured to contain there within one or more various contents 50. As is explained in greater detail below, the contents 50 of the reactor vessel 110 can include, but need not be limited to, a catalyst 51, a supercritical fluid 55, two or more reactants 66, and at least one product 88.

The catalyst 51, supercritical fluid 55, reactants 66, and product 88 may or may not be mutually exclusive. That is, in accordance with at least one embodiment of the present disclosure, at least a portion of the reactants 66 can also form at least a portion of the supercritical fluid 55. However, in accordance with another embodiment of the present disclosure, none of the reactants 66 form any portion of the supercritical fluid 55.

The reactor vessel 110 can be configured to contain a chemical reaction. Accordingly, the reactor vessel 110 can be configured to withstand extreme internal pressure, as well as extreme internal temperature. Additionally, the reactor vessel 110 can be configured to resist corrosion and other harsh effects generally associated with high temperature and/or high temperature and/or chemical reactions and the like.

The reactor vessel 110 can include at least one access opening 111, 112. The access opening 111, 112 can allow contents 50 to be placed into, and removed from, the reactor vessel 110. The reactor vessel 110 can include a first opening 111, and a second opening 112. In accordance with at least one embodiment of the present disclosure, the first opening 111 can be configured as an inlet opening, while the second opening 112 can be configured as an outlet opening, as is depicted.

The apparatus 100 can additionally include a pressure/temperature control system 120. The pressure/temperature control system 120 can be configured to control the pressure and/or the temperature of the contents 50 of the reactor vessel 110. The pressure/temperature control system 120 can be configured to control the pressure and/or the temperature of the reactants 66 before and/or after the reactants enter the reactor vessel 110. Similarly, the pressure/temperature control system 120 can be configured to control the pressure and/or the temperature of the product 88 before and/or after the product exits the reactor vessel 110. Additionally, the pressure/temperature control system 120 can be configured to affect one or more flow characteristics of the reactants 66 and/or product 88, as is discussed in greater detail below.

The pressure/temperature control system 120 can be configured to add heat energy to one or more contents 50, including but not limited to the catalyst 51, the supercritical fluid 55, the reactants 66 (before and/or after entering the reactor vessel 110), and the product 88 (before and/or after exiting the reactor vessel). The pressure/temperature control system 120 can be configured to remove heat energy from one or more contents 50, including but not limited to the catalyst 51, the supercritical fluid 55, the reactants 66 (before and/or after entering the reactor vessel 110), and the product 88 (before and/or after exiting the reactor vessel).

The pressure/temperature control system 120 can be configured to pressurize and/or to depressurize one or more contents 50, including but not limited to the supercritical fluid 55, the reactants 66 (before and/or after entering the reactor vessel 110), and the product 88 (before and/or after exiting the reactor vessel).

The pressure/temperature control system 120 can be configured to affect one or more flow characteristics of the reactants 66 and/or the product 88. By way of example, the pressure/temperature control system 120 can be configured to control the flow rate into the reactor vessel 110 of the reactants 66 and/or to control the flow rate out of the reactor vessel of the product 88.

The pressure/temperature control system 120 can be configured to maintain one or more contents 50 of the reactor vessel 110 within a predetermined temperature range and/or within a predetermined pressure range. The pressure/temperature control system 120 can be configured to maintain the flow rate of the reactants 66 into the reactor vessel 110 and/or the flow rate of the product 88 out of the reactor vessel, within given respective ranges.

Furthermore, the pressure/temperature control system 120 can be configured to maintain the given temperature range and/or pressure range and/or flow rate range while one or more given reactions are occurring within the pressure vessel 110. Such reactions can include batch process reactions and/or continuous process reactions.

Accordingly, the pressure/temperature control system 120 can be substantially in the form of, and/or can include, various specific devices and/or components configured to add heat energy and/or to remove heat energy and/or to pressurize and/or to depressurize and/or to affect one or more flow characteristics of, one or more contents 50 of the reactor vessel 110, including but not limited to the supercritical fluid 55, the reactants 66, and the product 88.

It is to be understood that, in accordance with one or more various embodiments of the present disclosure, one or more of such devices and/or components can facilitate only pressure control, or only temperature control, or a combination of both pressure control and temperature control. It is to be further understood that the pressure/temperature control system 120 can, in accordance with at least one embodiment of the present disclosure, exist as a combination pressure/temperature control system, or as a plurality of substantially discrete and separate systems such as a pressure control system and a temperature control system.

The reactor vessel 110 together with the pressure/temperature control system 120 can be configured to cause at least a portion of the contents 50 of the pressure vessel to be converted to, and/or to exist in, a supercritical phase or state (i.e., as a “supercritical fluid” 55). That is, the reactor vessel 110 and/or the pressure/temperature control system 120 can be constructed and/or configured to have sufficient physical characteristics required to withstand extreme pressures and/or temperatures generally associated with supercritical fluids and/or to produce sufficient heat energy to cause at least a portion of the contents 50 to attain a supercritical phase.

The apparatus 100 can include a dispersal system 130. A primary function of the dispersal system 130 can be dispersal or distribution of at least a portion of the catalyst 51 in at least a portion of the supercritical fluid 55. More specifically, the dispersal of the catalyst 51 in the supercritical fluid 55 can be such that at least a portion of the reactants 66 can come into contact with at least a portion of the catalyst 51 while so dispersed within the supercritical fluid, whereby a reaction between the reactants is facilitated. The dispersal system 130 can operate to disperse the catalyst 51 in the supercritical fluid 55 by way of any of a number of various means in accordance with a one or more of a number of respective embodiments of the present disclosure.

It is to be understood that various components of the apparatus 100 as well as other apparatus described herein, including various subsystems such as the pressure/temperature control system 120 and/or the dispersal system 130, can include one or more specific devices such as, but not limited to, a pressure sensor, a temperature sensor, a flow meter, a flow regulator, a controller, a heat exchanger, a radiator, a condenser, an evaporator, a compressor, a pump, a pressure regulator, a valve (including for example, a metering valve, a pressure regulating valve, a throttling valve, a check valve, and the like), a flow restrictor, an orifice plate and the like.

By way of example, dispersal of the catalyst 51 in the supercritical fluid 55 can include forming a dispersoid by dispersing fine particles of catalyst 51 within the supercritical fluid 55. In accordance with at least one embodiment of the present disclosure, the contents 50 and/or the supercritical fluid 55 and/or the catalyst 51 can be agitated and/or mixed so as to disperse or suspend the catalyst in the supercritical fluid.

Examples of agitation and/or mixing of the supercritical fluid 55 and catalyst 51 so as to disperse the catalyst in the supercritical fluid include, but are not limited to, turbulent pipeline flow with or without baffling, mechanical agitation with an impeller with or without baffling, mechanical agitation with a gas or gases via, for example, jets located along a reactor length and/or diameter, wherein the gas or gases can include reactants, products, and/or inert substances.

In accordance with at least one embodiment of the present disclosure, dispersal of the catalyst 51 in the supercritical fluid 55 can be accomplished by way of metered, patterned distribution of the catalyst in laminar pipeline flow. It is to be understood that pipeline flow described herein can be supersonic or subsonic.

In accordance with at least one embodiment of the present disclosure, two or more reactants 66 can be placed into the reactor vessel 110, and can then react to form at least one product 88. More specifically, the reactants 66 can be placed into the reactor vessel 110, wherein the contents 50 of the reactor vessel includes at least a portion of the reactants as well as the catalyst 51. Pressure and/or temperature of the contents 50, including but not limited to the reactants 66, can be controlled by the pressure/temperature control system 120, whereby at least a portion of the contents is in a supercritical phase, or exists as a supercritical fluid 55.

The dispersal system 130 can cause at least a portion of the catalyst 51 to be dispersed or suspended in the supercritical fluid 55 by way of, but not limited to, any of the dispersal means described herein. The reactants 66 can be exposed to, or can come into contact with, at least a portion of the catalyst 51 after the catalyst has achieved a state of dispersion or suspension in the supercritical fluid 55. Such exposure and/or contact of the reactants 66 to the catalyst 51 can initiate or cause a chemical reaction between the reactants.

Contact of the reactants 66 with the catalyst 51 and/or with one another can be facilitated and/or enhanced by agitation and/or mixing of the contents 50 of the reactor vessel 110. Additionally, the pressure/temperature control system 120 can be operated to control various aspects of such a chemical reaction as described above by controlling one or more of the pressure and/or temperature and/or flow rate of the contents 50 and/or of the reactants 66 and/or product 88.

The product 88 can be formed as a result of the chemical reaction between the reactants 66. The product 88 can then be removed, or extracted, from the reactor vessel 110 and collected. The product 88 can be in substantially final form when removed from the reactor vessel 110. Alternatively, in accordance with at least one embodiment of the present invention, the product 88 can undergo further processing and/or refinement after being extracted from the reactor vessel 110.

The chemical reaction such as that described above, and which can occur within the reactor vessel 110, can be substantially a batch process reaction, or alternatively can be substantially a continuous process reaction. With continued reference to FIG. 2, and in accordance with one embodiment of the present disclosure, a batch process chemical reaction can be made to occur by first placing various substances such as the reactants 66 into the reactor vessel 110. The reactor vessel can then be sealed by closure of the openings 111, 112.

Upon sealing of the reactor vessel 110, the pressure/temperature control system 120 and/or the dispersal system 130 can be operated in the manner described above to facilitate the occurrence of a chemical reaction within the reactor vessel. That is, upon sealing the reactor vessel 110, the pressure/temperature control system 120 can be operated in a manner such that at least a portion of the contents 50 exists as a supercritical fluid 55, and the dispersal system 130 can be operated in a manner such that at least a portion of the catalyst 51 is dispersed or suspended in the supercritical fluid so as to facilitate the occurrence of a chemical reaction between the reactants to form a product 88.

In accordance with at least one embodiment of the present disclosure, the dispersal system 130 includes means of circulating at least a portion of the contents 50 through and/or around the reactor vessel 110 whereby at least a portion of the catalyst 51 is dispersed or suspended in the supercritical fluid 55. That is, such circulation of the contents 50 of the reactor vessel 110 can cause the supercritical fluid 55 to exist in a turbulent state that is sufficient for at least a portion of the catalyst 51 to be dispersed or suspended within the supercritical fluid.

Upon substantial completion of the batch process chemical reaction, one or more of the openings 111, 112 can be unsealed and the product 88 can be extracted or removed from the reactor vessel 110. In accordance with at least one embodiment of the present disclosure, the pressure/temperature control system 120 can be operated to cool and/or depressurize the contents 50 after substantial completion of the chemical reaction to facilitate removal of the product 88 from the reactor vessel 110 and/or to facilitate ease of handling and/or collection of the product.

With continued reference to FIG. 2, and in accordance with another embodiment of the present disclosure, a continuous process chemical reaction can be made to occur within the reactor vessel 110, which can contain a catalyst 51. The reactor vessel 110 can also contain contents, or mixture, 50 that can include, by way of example, one or more reactants 66 such as hydrogen and/or carbon monoxide, as well as other substances such as water and/or carbon dioxide, or the like. The pressure/temperature control system 120 can be operated in a manner such that at least a portion of the contents, or mixture, 50 exists as a supercritical fluid 55, while the dispersal system 130 can be operated to cause at least a portion of the catalyst 51 to be dispersed or suspended in the supercritical fluid.

The continuous process chemical reaction can proceed with injection or addition of a substantially continuous stream of reactants 66 into the reactor vessel 110 by way of the inlet opening 111. In accordance with at least one embodiment of the present disclosure, the dispersal system 130 includes means of injecting the reactants 66 into the reactor vessel 110 in a manner whereby the contents 50 are agitated and/or mixed, and wherein such agitation or mixing of the contents can cause the supercritical fluid 55 to exist in a turbulent state that facilitates dispersal and/or suspension of at least a portion of the catalyst 51 within the supercritical fluid.

Means of injecting the reactants 66 into the reactor vessel 110 include pressurizing the reactants 66 by way of a pump or compressor (not shown) and releasing the pressurized reactants into the reactor vessel through one or more nozzles (not shown) to create one or more high velocity streams of reactant within the reactor vessel. It is to be understood, however, that injection of the reactants is not required to practice the invention.

The continuous process chemical reaction can continue by removing, or bleeding, a continuous stream from the outlet 112 of the reactor vessel 110. The stream can include, but is not necessarily limited to, a portion of the product 88, a portion of one or more reactants 66, a portion of the catalyst 51, and/or a portion of the supercritical fluid 55. Substances other than the product 88 can be removed from the product in accordance with one or more various recovery, filtration, purification, and/or separation processes, including one or more processes known to those of ordinary skill in the art, and which are therefore not discussed further.

Reactants 66 and/or catalyst 51, as well as other substances and/or materials that are recovered and/or otherwise removed from the stream of product 88 can be routed back into the reactor vessel 110. Moreover, after product 88 in the product stream exits the reactor vessel, such product can be further processed, which can include, for example, cooling, depressurizing, refining, purifying, and the like.

In accordance with one or more embodiments of the present disclosure, the catalyst 51 and/or the reactants 66 and/or the product 88 can be specifically configured as described below. That is, in accordance with one or more embodiments of the present disclosure, the catalyst 51 can be a transition metal catalyst selected from Group VI metals. The catalyst 51 can be a sulfided transition metal catalyst selected from Group VI metals.

The catalyst 51 can be substantially molybdenum based. That is, the catalyst 51 can consist of substantially molybdenum, or can consist substantially of molybdenite, or sulfided molybdenum, or can be substantially molybdic, or can be substantially molybdous. It is understood that the catalyst 51 need not be molybdenum based in order to practice the invention. That is, in accordance with various alternative embodiments of the present disclosure, the catalyst 51 can consist substantially of copper and/or zinc oxide and/or alumina, as well as other elements and compounds.

Regardless of the specific type or material content, the catalyst 51 can be sulfided, and/or can be substantially particulate. The term “particulate” is intended to encompass any particle size that is capable of being dispersed or suspended in any given supercritical fluid 55 by way of any of the means described herein. Moreover, the catalyst 51 can be activated and/or can be alkali-doped.

By way of example, the reactants 66 can be hydrogen and carbon monoxide. The reactants 66 can be hydrogen and carbon monoxide that are included in a mixture of other substances. By way of further example, the reactants 66 can be hydrogen and carbon monoxide that are included in synthesis gas, or syngas. It is to be understood, however, that the reactants 66 need not be limited to hydrogen and carbon monoxide in order to practice the invention. That is, in accordance with alternative embodiments of the disclosure, other substances can be used as reactants 66.

If the reactants 66 are substantially hydrogen and carbon monoxide, then the product 88 can include one or more specific forms or types of alcohol. By way of example, the product 88 can include one or more of high-rank alcohols and low-rank alcohols. By way of further example, high-rank alcohols can include butanol, ethanol, hexanol, propanol, and the like, while low-rank alcohols can include methanol. However, it is to be understood that the product 88 is not required to be any form of alcohol in order to practice the invention.

In accordance with at least one embodiment of the present disclosure, a method of producing alcohol includes providing a mixture that includes carbon monoxide and hydrogen. The mixture is characterized by a temperature and a pressure. The mixture can be contained within a reactor vessel such as the reactor vessel 110, which is described herein with respect to the accompanying figures.

The method further includes controlling the temperature and/or pressure of the mixture so that at least a portion of the mixture exists as a supercritical fluid (i.e., a fluid in a supercritical phase). Control of the temperature and/or pressure of the mixture can be accomplished by way of the pressure/temperature control system 120, which is described herein with respect to the accompanying figures.

The method also includes dispersing a catalyst the supercritical fluid. Dispersal of the catalyst 51 can be accomplished by way of the dispersal system 130, which is described herein with respect to the accompanying figures. Dispersal of the catalyst 51 can be accomplished by any of a number of means of catalyst dispersal, including those means described herein.

The method includes causing at least a portion of the carbon monoxide and the hydrogen to contact the dispersed catalyst, whereby a reaction occurs to produce the alcohol. The reaction can be initiated and/or aided by exposure of the carbon monoxide and the hydrogen to one another and to the catalyst that is dispersed, suspended, or mixed in the supercritical fluid. The reaction can produce one or more types of alcohol. Dispersal of the catalyst can be facilitated by, but need not be limited to, agitation of the mixture and circulation of the mixture.

It is noted that various methods of producing alcohol in accordance with the respective embodiments of the present disclosure are distinguished from other prior art methods, such as that disclosed by Jackson et al. in U.S. Pat. Nos. 6,753,353 and 6,248,796. Specifically, Jackson et al. teach suspension of a catalyst in a liquid carrier to form a liquid catalyst slurry, whereas methods in accordance with the various embodiments of the present disclosure teach dispersal of a catalyst in a supercritical fluid. It has been found that means disclosed herein, which employ catalyst dispersed in a supercritical fluid, result in increased reaction efficiency, including increased reaction rates.

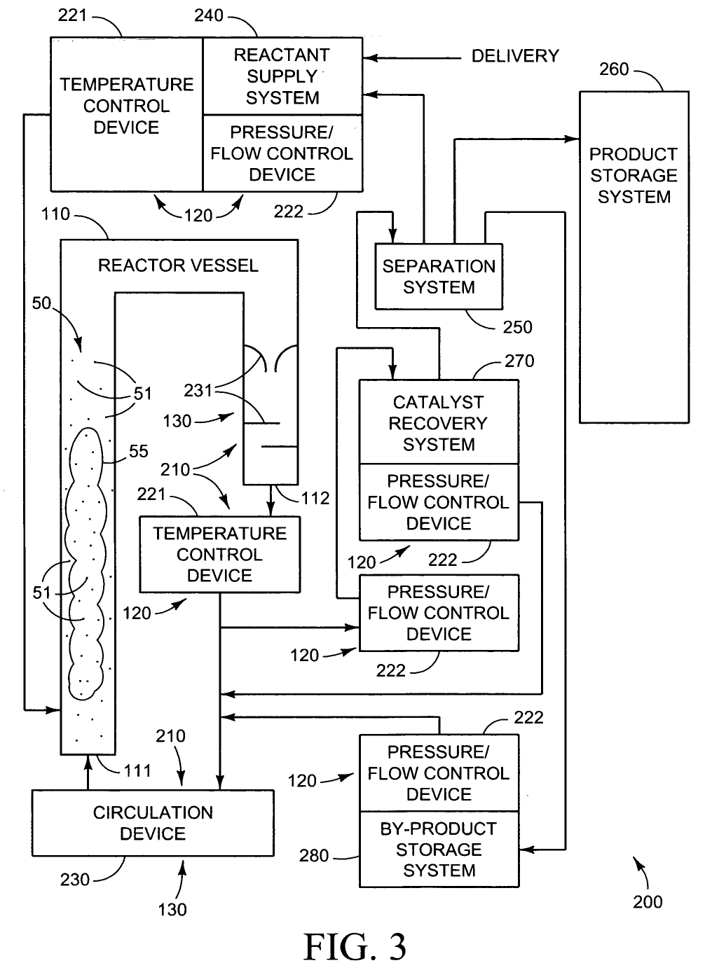

Turning now to FIG. 3, a schematic diagram is shown in which an apparatus 200 is depicted in accordance with at least one embodiment of the present disclosure. The apparatus 200 can include a reactor system 210. In accordance with at least one embodiment of the present disclosure, the reactor system 210 can be substantially in the form of a circuitous reactor system, wherein the reactor system is substantially in the form of a continuous circuit. The reactor system 210 is configured to contain and process the contents 50, which contents can include but need not be limited to the catalyst 51, and wherein at least a portion of the contents can exist as a supercritical fluid, as is explained above.

The reactor system 210 can include the reactor vessel 110, which has been described above with reference to FIG. 2. The reactor vessel 110 can have any of a number of specific shapes and/or configurations in accordance with any of a number of respective embodiments of the present disclosure. By way of example, a study of FIG. 3 reveals that in accordance with one embodiment of the present disclosure the reactor vessel 110 can include and/or can be substantially in the form of an elongated pipe. The reactor vessel 110, when substantially in the form of an elongated pipe, can follow any of a number of possible paths. For example, the reactor vessel 110 can be substantially straight, or can include a number of curves, and the like. Moreover, the reactor vessel 110, as well as other various components of the apparatus 200, can have any of a number of possible orientations relative to gravity.

The reactor system 210 can include pressure/temperature control system 120, which is described above with respect to FIG. 2. With continued reference to FIG. 3, the pressure/temperature control system 120 can include at least one temperature control device 221. As is depicted in FIG. 3, the pressure control system 120 can include two temperature control devices 221 in accordance with at least one embodiment of the present disclosure. The function of the temperature control devices 221 are described in greater detail below.

The temperature control device 221 can include and/or can be substantially in the form of, by way of example, a heat exchanger. A study of FIG. 3 reveals that the pressure/temperature control system 120 can include additional components. Such additional components can include one or more pressure/flow control devices 222. The pressure/flow control device 222 can be configured to control pressure and/or flow rate and/or flow direction, as is described in greater detail below.

The reactor system 200 can include a dispersal system 130, which is described above with respect to FIG. 2. With continued reference to FIG. 3, the dispersal system 130 can include a circulation device 230. The circulation device 230 can form an integral part of the reactor system 210. The dispersal system 130 can include one or more flow devices 231. By way of example, the flow device 231 can be in the form of, but need not be limited to, a baffle, a nozzle, a venturi, an orifice plate, and the like.

The reactor vessel 110 can be fluidically connected between the circulation device 230 and the temperature control device 221, and the temperature control device can be fluidically connected between the reactor vessel and the circulation device, while the circulation device can be fluidically connected between the temperature control device and the reactor vessel so as to form a continuous loop, or circuit, as is depicted. It is to be understood, however, that the various components of the reactor system 110 need not be arranged in the order and/or configuration specifically described herein to practice the invention.

The circulation device 230 can be configured to circulate the contents 50 around such a loop or circuit formed by the reactor system 210. That is, by way of example and in accordance with one embodiment of the present disclosure, the circulation system 230 can impart mechanical energy to the contents 50 of the reactor system 210 so as to cause the contents to move in a substantially continuous circulatory manner through the reactor system.

By way of example, the circulation device 230 can include and/or can be substantially in the form of a pump and/or compressor that is configured to cause (for example, by pumping) at least a portion of the contents 50 to flow through the reactor vessel 110 to the temperature control device 221, and through the temperature control device to the circulation device, and through the circulation device back to the reactor vessel in a substantially continuous manner.

The circulation device 230 can be configured to facilitate dispersal of the catalyst 51 in the supercritical fluid 55, which is described above. That is, by way of example, the circulation device 230 can be configured to circulate at least a portion of the contents through at least a portion of the reactor system 210 at a flow rate sufficient to cause the supercritical fluid 55 to exist in a substantially turbulent state that is, in turn, sufficient to cause at least a portion of the catalyst 51 to be suspended in the supercritical fluid. It is to be understood however, that other means of dispersal of the catalyst 51 within the supercritical fluid 55 are contemplated in accordance with various respective embodiments of the present disclosure, as is explained above.

Various characteristics of one or more components of the reactor system 210 can be configured to affect one or more parameters of a reaction that takes place within the reactor system. For example, in accordance with one embodiment of the present disclosure the reactor vessel 50 of the apparatus 200 can be substantially in the form of an elongated pipe, while the circulation device 230 can be substantially in the form of a pump, compressor, turbine, or the like.

In accordance with such an embodiment, the pipe diameter and/or length, as well as the pumping capacity of the pump, can be determined so as to increase the probability that the flow characteristics of the supercritical fluid include a level of turbulence sufficient to disperse and/or suspend the catalyst 51 in the supercritical fluid. Additionally, one or more flow devices 231 can be employed within the reactor vessel 110, or within various connecting pipes (not referenced), to affect the flow characteristics of the contents 50 and/or the supercritical fluid 55. Moreover, the length of the pipe can be determined so as to increase the probability that a desired level of reaction time within the pipe is achieved in view of a given flow rate.

With continued reference to FIG. 3, the apparatus 200 can include at least one reactant supply system 240. The reactant supply system 240 can be configured to store one or more reactants, such as the reactants 66 described above with reference to FIG. 2. With continued reference to FIG. 3, the reactant supply system 240 can be configured to supply one or more reactants to the reactor system 210. Accordingly, the reactant supply system 240 can be fluidically connected to the reactor system 210 as depicted.

A pressure/flow control device 222 can be associated with the reactant supply system 240. The pressure/flow control device 222 can be configured to first receive one or more reactants from the reactant supply system 240 and then to pressurize the reactants to a degree required for injection of the reactants into the reactor system 210. That is, in accordance with at least one embodiment of the present disclosure, it is understood that pressurization of the reactants to a level greater than the pressure within the reactor system 210 is be required for injection of the reactants into the reactor system.

In accordance with at least one embodiment of the present disclosure, the pressure/flow control device 222 associated with the reactant supply system 240 can be configured to regulate and/or meter the mass and/or volume of reactants injected into the reactor system 210. Such regulating and/or metering can be controlled as a function of various parameters such as, but not limited to, reaction start-up mode, reaction temperature, reaction pressure, chemical analysis of the reactants and/or products, and the like.

By way of example, the apparatus 200 can be configured such that the reactants can be pressurized by an associated pressure/flow control device 222 so as to be injected into the reactor vessel 110 as depicted. By way of further example, the pressure/flow control device 222 can include and/or can be substantially in the form of one or more of a pump, a compressor, a metering valve, a pressure regulating valve, a flow regulator, a check valve, a flow meter, a flow restrictor, an orifice plate, a sensor, a controller, and the like.

A temperature control device 221 can also be associated with the reactant supply system 240, as is depicted. The temperature control device 221 that is associated with the reactant supply system 240 can be configured to control the temperature of the reactants before the reactants enter the reactor vessel 110 and/or the reactor system 210.

By way of example, the temperature control system 221 associated with the reactant supply system 240 can be configured to heat the reactants after the reactants are released from the reactant supply system and before the reactants enter the reactor vessel. In such a manner the temperature control system 221 associated with the reactant supply system 240 can be employed to control, at least in part, the temperature of the contents 50 of the reactor system 210.

With continued reference to FIG. 3, the apparatus 200 can include a separation system 250 that is fluidically connected to the reactor system 210 as depicted. The separation system 250, as well as other components discussed further below, can be fluidically connected to the reactor system 210 in the manner of a bleed-off leg or the like, whereby a bleed stream consisting of a portion of the contents 50 of the reactor system is bled from the reactor system and then routed to the separation system.

It is to be understood that, in accordance with any of a number of various embodiments of the present disclosure, the separation system 250 can be connected to the reactor system 210 at any of a number of respective locations. By way of example, the separation system 250 can be connected to the reactor system 210 substantially between the temperature control device 221 and the circulation device 230, as is depicted in FIG. 3. Alternatively, by way of example, the separation system 250 can be connected to the reactor system 210 at a location between the reactor vessel 110 and the temperature control device 221.

The separation system 250 can be configured to separate and/or recover the desired product from the remainder of the contents 50 of the reactor system 210. The separation system 250 can be configured to perform any of a number of specific processes required to recover the desired product. By way of example, the separation system 250 can be configured to perform a distillation process in accordance with which the desired product is recovered.

The separation system 250 can be further configured to separate and/or recover other substances from the bleed stream. By way of example, the separation system 250 can be configured to separate and/or recover residual reactants from the bleed stream, and can also be configured to send such residual reactants back to the reactant supply system 240 as depicted. By way of further example, the separation system 250 can be configured to separate and/or recover one or more by-products from the bleed stream.

Still referring to FIG. 3, the apparatus 200 can include a catalyst recovery system 270. The catalyst recovery system 270 can be fluidically connected between the reactor system 210 and the separation system 250. The catalyst recovery system 270 can be configured to separate and/or recover catalyst 51 from the bleed stream before the bleed stream reaches the separation system 250. The catalyst recovery system 270 can be configured to clean and/or to reactivate the catalyst.

The catalyst recovery system 270 can be configured to return recovered catalyst 51 to the reactor system 210 as depicted. Such return of recovered catalyst 51 can be accomplished by way of an associated pressure/flow control device 222. By way of example, the catalyst recovery system 270 can be in the form of and/or can include one or more of a heat exchanger, a flash tank, a pump, a decanter, a filtration device, a settling tank, a make up tank, a tank agitator, a centrifuge, a cleaning device, and the like.

In accordance with at least one embodiment of the present disclosure, the catalyst recovery system 270 can be configured to rejuvenate at least a portion of the recovered catalyst 51 by adding a doping agent to the recovered catalyst before the recovered catalyst is returned to the reactor system 210. By way of example only, the doping agent can be an alkali doping agent. In accordance with at least one embodiment of the present disclosure, the catalyst recovery system 270 can be configured to collect and dispose of at least a portion of the recovered and/or spent catalyst, and to add new and/or previously rejuvenated catalyst 51 to the reactor system 210.

The apparatus 200 can also include a pressure/flow control device 222 that is fluidically connected between the reactor system 210 and the catalyst recovery system 270 as depicted. Such a pressure/flow control device 222 can be configured to assist in controlling the reaction pressure and/or the pressure within the reactor system 210 by controlling the bleed stream flow rate and/or pressure and by preventing backflow and the like.

The apparatus 200 can include a product storage system 260. The product storage system is configured to store the product after the product is recovered from the bleed stream by the separation system 250 as described above. Accordingly, the product storage system 260 can be fluidically connected to the separation system 250 as depicted, whereby the recovered product can be routed from the separation system to the product storage system for storage.

The apparatus can also include a by-product storage system 280. The by-product storage system 280 can be configured to store one or more by-products that can be separated and/or recovered from the bleed stream as described above. Accordingly, the by-product storage system 280 can be fluidically connected to the separation system 250 as depicted, whereby a given by-product can be routed to the by-product storage system from the separation system. The by-product storage system 280 can be connected to the reactor system 210 by way of an associated pressurization device 222 as depicted, whereby a predetermined quantity of the given by-product can be routed into the reactor system.

Still referring to FIG. 3, the reactant supply system 240 can be configured to store hydrogen and/or carbon monoxide. By way of example, the reactant supply system 240 can be configured to store a mixture such as syngas or the like that contains both hydrogen and carbon monoxide. The pressure/flow control device 222 associated with the reactant supply system 240 can be substantially in the form of a compressor to pressurize the mixture, such as syngas, for injection into the reactor vessel 110. The temperature control device 221 associated with the reactant supply system 240 can be substantially in the form of a heat exchanger to heat the hydrogen and carbon monoxide after being released from the reactant supply system and before entering the reactor system 210.

With continued reference to FIG. 3, the reactor vessel 110 can contain, by way of example, a particulate catalyst 51, which can be molybdenum based. The circulation device 230 can cause the contents 50, which can include a mixture such as syngas and the catalyst 51, to be circulated around the reactor system 210. The pressure/temperature control system 120 can be operated to cause at least a portion of the contents or mixture 50, which can include syngas, to exist as a supercritical fluid 55.

Circulation and/or flow of the contents or mixture 50 through at least the reactor vessel 110 can cause at least a portion of the supercritical fluid 55 to exist in a state of turbulence sufficient to cause at least a portion of the catalyst 51 to be dispersed and/or suspended in the supercritical fluid. During circulation and/or flow of the mixture, including syngas, through the reactor vessel 110, the hydrogen and carbon monoxide in the syngas can be exposed to one another and can be exposed to the catalyst 51. Exposure of the hydrogen and carbon monoxide to one another and to the catalyst 51 can occur with the catalyst being in a state of dispersion within the supercritical fluid 55.

Such exposure of the hydrogen and carbon monoxide to the catalyst 51 and to one another while the catalyst is in a state of dispersion within the supercritical fluid 55 can result in a chemical reaction to produce a product such as one or more types of alcohol.

A bleed stream can be tapped from the reactor system 210 and fed through the pressure/flow control device 222 as is depicted, and then through the catalyst recovery system 270, and then into the separation system 250. The bleed stream can include the one or more products, as well as catalyst, as well as one or more components of the mixture, including syngas. Substantially all of the catalyst 51 can be removed and/or recovered from the bleed stream. The recovered catalyst 51 can be processed and/or returned to the reactor system 210 as is described above.

In accordance with at least one embodiment of the present disclosure, the separation system 250 can separate and/or recover high rank alcohol such as, for example, butanol, ethanol, hexanol, and/or propanol, from the bleed stream and can send the separated high rank alcohol to the product storage system 260. The separation system can also separate and/or recover low rank alcohol, such as, for example, methanol, from the bleed stream and can return the low rank alcohol to the reactor system 210 by way of the associated pressurization device 222. The separation system 250 can additionally separate and/or recover residual syngas (e.g. hydrogen and/or carbon monoxide) from the bleed stream and can send the recovered syngas to the reactant supply system 240.

With reference to FIGS. 2 and 3, in accordance with at least one embodiment of the present disclosure, one or more conditions within the reactor vessel 110 and/or within the reactor system 210 can be controlled such that substantially all of the reaction between the reactants is caused to occur within the supercritical fluid 55. That is, in accordance with at least one embodiment of the present disclosure, the flow rate and/or the velocity and/or the temperature and/or the pressure of at least some of the contents 50 can be controlled in a manner whereby a given portion of the contents is maintained as a supercritical fluid, and wherein the given portion is sufficient to allow substantially all of the reaction between the reactants to occur within the supercritical fluid.

In accordance with at least one other embodiment of the present disclosure, one or more conditions within the reactor vessel 110 and/or within the reactor system 210 can be controlled such that the reaction between the reactants initiates or starts within the supercritical fluid 55, and at least a portion of the reaction between the reactants occurs within the supercritical fluid while the remainder of the reaction occurs outside the supercritical fluid.

That is, in accordance with at least one other embodiment of the present disclosure, the flow rate and/or the velocity and/or the temperature and/or the pressure of at least some of the contents 50 can be controlled in a manner whereby a given portion of the contents is maintained as a supercritical fluid 55 while a remainder of the contents exists as a gas and/or as a liquid, and wherein the given portion is sufficient to allow the reaction between the reactants to initiate within the supercritical fluid, and to allow at least a portion of the reaction to occur within the supercritical fluid while another portion of the reaction occurs outside the supercritical fluid.

In accordance with one embodiment of the present disclosure, a method of producing alcohol includes providing a reactor and allowing to flow into the reactor a substance that includes carbon monoxide and hydrogen. The substance containing hydrogen and carbon monoxide can be, for example, syngas. The reactor can be, for example, the reactor 110, which can form at least a portion of the reactor system 210, which is described herein with respect to FIG. 3.

The method further includes containing within the reactor a mixture that includes the substance. That is, the mixture can include matter in addition to the substance. The mixture can be characterized by a pressure and a temperature. That is, a pressure and a temperature of the mixture can be detected and measured.

The method includes controlling the pressure and/or the temperature of the mixture so that at least a portion of the mixture exists as a supercritical fluid. The pressure and/or the temperature of the mixture can be controlled by way of the pressure/temperature control system 120, which is described herein with respect to the accompanying figures.

The method includes dispersing within the supercritical fluid a substantially particulate catalyst and causing at least a portion of the carbon monoxide and the hydrogen to contact the catalyst, whereby a reaction occurs between the carbon monoxide and the hydrogen to produce the alcohol. The catalyst can be dispersed within the supercritical fluid by way of the dispersal system 130, which is described herein with respect to the accompanying figures.

The method further includes allowing to flow out of the reactor a bleed stream containing at least a portion of the alcohol produced from the reaction. The step of allowing a bleed stream to flow out of the reactor can, by way of example, be accomplished by allowing a bleed stream to flow from the reactor system 210 (shown in FIG. 3) as is described above with respect to FIG. 3. The method can include recovering catalyst from the bleed stream, and can further include returning the recovered catalyst to the reactor.

The method can include separating from the bleed stream carbon monoxide and/or hydrogen. Such separated carbon monoxide and/or hydrogen can be returned to the reactor in accordance with at least one embodiment of the present disclosure. The method can also include separating from the bleed stream a by-product, and the method can further include returning at least a portion of the by-product to the reactor. The by-product can be a type of alcohol, and more specifically, can be low rank alcohol.

The method can include circulating the mixture around the reactor to facilitate dispersal of the catalyst. By way of example, the reactor can be the reactor 110, which forms a portion of the reactor system 210 as described herein with respect to FIG. 3. Thus, in accordance with the method, circulation of the mixture can be accomplished by employing the circulation device 230 (shown in FIG. 3) to circulate the mixture around the reactor system.

The method can include causing at least a portion of the mixture to flow through the reactor in a substantially turbulent manner, which can be for the purpose of facilitating dispersal of the catalyst in the supercritical fluid. The method can include causing at least a portion of the mixture to flow through the reactor in a substantially laminar manner, which can be for the purpose of facilitating dispersal of the catalyst in the supercritical fluid.

The method can include causing the at least a portion of the mixture to flow through the reactor supersonically, which can be for the purpose of facilitating dispersal of the catalyst in the supercritical fluid. In accordance with at least one embodiment of the present disclosure, the method includes injecting into the reactor a stream of fluid to facilitate dispersal of the catalyst within the supercritical fluid. Such an injection stream can be at a velocity sufficient to substantially disperse at least a portion of the catalyst in at least a portion of the supercritical fluid.

In accordance with at least one embodiment of the present disclosure, the stream of fluid that is injected into the reactor is made up substantially of the substance that includes the carbon monoxide and the hydrogen. In accordance with another embodiment of the present disclosure, the stream of fluid is made up of substantially inert matter. In accordance with yet another embodiment of the present disclosure, the stream of fluid is made up of substantially alcohol.

In accordance with another embodiment of the present disclosure, a method of producing alcohol includes dispersing a catalyst in a supercritical fluid and causing carbon monoxide and hydrogen to contact the dispersed catalyst, whereby a reaction occurs to produce the alcohol. That is, in accordance with the method, carbon monoxide and hydrogen can react with one another in the presence of the dispersed catalyst to produce one or more types of alcohol.

In accordance with at least one embodiment of the present invention, a method of producing alcohol includes causing the reaction to occur substantially within the supercritical fluid. In accordance with at least one other embodiment of the present invention, a method of producing alcohol includes causing the reaction to initiate within the supercritical fluid and causing at least a portion of the reaction to occur within the supercritical fluid while causing the remainder of the reaction to occur outside of the supercritical fluid.

The preceding description has been presented only to illustrate and describe exemplary methods and apparatus of the present invention. It is not intended to be exhaustive or to limit the disclosure to any precise form disclosed. Many modifications and variations are possible in light of the above teaching. It is intended that the scope of the invention be defined by the following claims.

What is claimed is:

1. A method of producing alcohol, comprising:
providing a reactor;
allowing to flow into the reactor a substance comprising carbon monoxide and hydrogen;
containing within the reactor a mixture comprising the substance, wherein the mixture is characterized by a temperature and a pressure;
controlling the temperature and the pressure, whereby at least a portion of the mixture exists as a supercritical fluid;
dispersing within the supercritical fluid a substantially particulate catalyst;
causing at least a portion of the carbon monoxide and hydrogen to contact the catalyst, whereby a reaction occurs to produce the alcohol; and
allowing to flow from the reactor a bleed stream comprising at least a portion of the alcohol.

2. The method of claim 1, further comprising causing the reaction to occur substantially within the supercritical fluid.

3. The method of claim 1, wherein dispersing the catalyst comprises circulating the mixture around the reactor to facilitate dispersal of the catalyst.

4. The method of claim 1, wherein dispersing the catalyst comprises causing at least a portion of the mixture to flow through the reactor in a substantially turbulent manner to facilitate dispersal of the catalyst.

5. The method of claim 1, wherein dispersing the catalyst comprises causing at least a portion of the mixture to flow through the reactor in a substantially laminar manner to facilitate dispersal of the catalyst.

6. The method of claim 1, wherein dispersing the catalyst comprises causing at least a portion of the mixture to flow through the reactor substantially supersonically, to facilitate dispersal of the catalyst.

7. The method of claim 1, wherein dispersing the catalyst comprises injecting into the reactor a stream of fluid to facilitate dispersal of the catalyst.

8. The method of claim 7, wherein the stream of fluid comprises substantially the substance.

9. The method of claim 7, wherein the stream of fluid comprises substantially inert matter.

10. The method of claim 7, wherein the stream of fluid comprises substantially alcohol.

11. The method of claim 1, wherein allowing to flow into the reactor the substance comprises injecting into the reactor a stream of the substance to facilitate dispersal of the catalyst.

12. The method of claim 1, further comprising:
recovering catalyst from the bleed stream; and
returning the recovered catalyst to the reactor.

13. The method of claim 1, further comprising:
separating from the bleed stream carbon monoxide and hydrogen; and
returning the separated carbon monoxide and hydrogen to the reactor.

14. The method of claim 1, further comprising:
separating from the bleed stream low rank alcohol; and
returning at least a portion of the low rank alcohol to the reactor.

15. Alcohol produced according to the method of claim 1.

16. A method of producing alcohol, comprising:
providing a mixture comprising carbon monoxide and hydrogen, wherein the mixture is characterized by a temperature and a pressure;
controlling the temperature and the pressure, whereby at least a portion of the mixture exists as a supercritical fluid;
dispersing a catalyst within the supercritical fluid; and
causing at least a portion of the carbon monoxide and hydrogen to contact the dispersed catalyst, whereby a reaction occurs to produce the alcohol.

17. The method of claim 16, further comprising causing the reaction to occur substantially within the supercritical fluid.

18. The method of claim 16, wherein dispersing the catalyst comprises agitating the mixture to facilitate dispersal of the catalyst.

19. The method of claim 16, wherein dispersing the catalyst comprises circulating the mixture to facilitate dispersal of the catalyst.

20. The method of claim 16, wherein the supercritical fluid is substantially reactive.

21. The method of claim 16, wherein the catalyst is a transition metal catalyst selected from Group VI metals.

22. The method of claim 16, wherein the catalyst is a sulfided transition metal catalyst selected from Group VI metals.

23. The method of claim 16, wherein the catalyst is substantially molybdenum based.

24. The method of claim 16, wherein the catalyst comprises substantially molybdenum.

25. The method of claim 16, wherein the catalyst comprises substantially molybdenite.

26. The method of claim 16, wherein the catalyst is substantially molybdic.

27. The method of claim 16, wherein the catalyst is substantially molybdous.

28. The method of claim 16, wherein the catalyst is sulfided.

29. The method of claim 16, wherein the catalyst is substantially particulate.

30. The method of claim 16, wherein the mixture comprises substantially syngas.

31. Alcohol produced according to the method of claim 16.

32. A method of producing alcohol, comprising:
dispersing a catalyst in a supercritical fluid; and
causing carbon monoxide and hydrogen to contact the dispersed catalyst, whereby a reaction occurs to produce the alcohol.

33. Alcohol produced according to claim 32.
